(19) United States
(12) Patent Application Publication
(10) Pub. No.: US 2018/0168663 A1
(43) Pub. Date: Jun. 21, 2018

(54) Windlass Tourniquet With Embedded Writing Implement

(71) Applicant: Misty A. Hill, Rockwall, TX (US)

(72) Inventor: Misty A. Hill, Rockwall, TX (US)

(21) Appl. No.: 15/382,898

(22) Filed: Dec. 19, 2016

(51) Int. Cl. A61B 17/132

(52) U.S. Cl. CPC A61B 17/1327

(57) ABSTRACT

A tourniquet comprising: i) a strap for encircling a limb; and ii) a tightening rod for tightening the strap around the limb, wherein the tightening rod includes a first writing tool. The tightening rod is cylindrical in shape and the first writing tool is disposed internally to the tightening rod and held in place by means of screw threading. Additionally, the tightening rod may further include a second writing tool. The second writing tool is inserted in a second end of the tightening rod opposite the first end and held in place by means of screw threading.

### TECHNICAL FIELD

The present application relates generally to windlass tourniquets and, more specifically, to a windlass tourniquet in which a writing tool is implemented in the rod used to tighten the tourniquet.

### BACKGROUND

A tourniquet is a cuff-like emergency medical device used to stop severe bleeding prior to bringing an injured person to a hospital. The tourniquet is wrapped around an injured limb near the location of the wound and tightened until the blood vessels below the tourniquet are squeezed shut by the pressure. Tourniquets have been used to save lives in critical situations with comparatively low risk of injury. Prompt application of a tourniquet before patients go into shock results in higher survival rates than instances where tourniquets were not used or were used after a delay.

One well-known type of tourniquet is the windlass tourniquet, which incorporates a tightening rod that is enmeshed with a band in the tourniquet. The rod is rotated or twisted to tighten the band around the injured limb. When the rod has been turned sufficiently to halt bleeding, the rod is typically locked in place by a clip or similar apparatus that prevents the rod from unwinding when the person applying the tourniquet releases the rod. Exemplary windlass tourniquets are disclosed in U.S. Pat. No. 7,842,067, U.S. Pat. No. 8,652,164, U.S. Pat. No. 8,926,651, and U.S. Pat. No. 9,456,826. The disclosures of each one of U.S. Pat. No. 7,842,067, U.S. Pat. No. 8,652,164, U.S. Pat. No. 8,926,651, and U.S. Pat. No. 9,456,826 are hereby incorporated into the present application as if fully set forth herein.

One commercially available windlass tourniquet is the Combat Application Tourniquet (CAT)®, which incorporates a tightening rod that locked in place by a C-shaped clip to prevent the rod from unwinding. Furthermore, a closing strap covers the opening of the C-shaped clip after the rods in locked in place. The closing strap is held in place by Velcro strips on the closing strap and the exterior of the C-shaped clip.

Advantageously, the closing strap has a white label on the exterior with the text “TIME:” printed thereon. This enables the first responder or another person to write on the white label the time at which the tourniquet was applied. This is important because the tourniquet must be removed in six (6) hours, which prevents permanent damage to the limb due to extended lack of blood circulation. However, in many emergency situations, the first responder or another person may not have a pen, pencil, marker, or other writing implement at hand. This reduces the value of having the TIME: label on the tourniquet. Therefore, there is a need in the art for an improved windlass tourniquet.

### SUMMARY

To address the above-discussed deficiencies of the prior art, it is a primary object to provide a tourniquet comprising: i) a strap for encircling a limb; and ii) a tightening rod for tightening the strap around the limb, wherein the tightening rod includes a first writing tool.

In one embodiment, the tightening rod is cylindrical in shape and the first writing tool is disposed internally to the tightening rod and held in place by means of screw threading.

In another embodiment, the tightening rod further includes a second writing tool.

In still another embodiment, the second writing tool is inserted in a second end of the tightening rod opposite the first end and held in place by means of screw threading.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a tourniquet in accordance with an embodiment of the disclosure.

FIG. 2 illustrates the tourniquet in FIG. 1 in further detail.

FIG. 3 illustrates the tourniquet in FIG. 1 applied to the limb of a patient.

FIG. 4 illustrates the tourniquet in FIG. 3 in further detail.

FIG. 5 illustrates in greater detail the tightening rod of the tourniquets in FIGS. 1-4 according to an embodiment of the disclosure.

### DETAILED DESCRIPTION

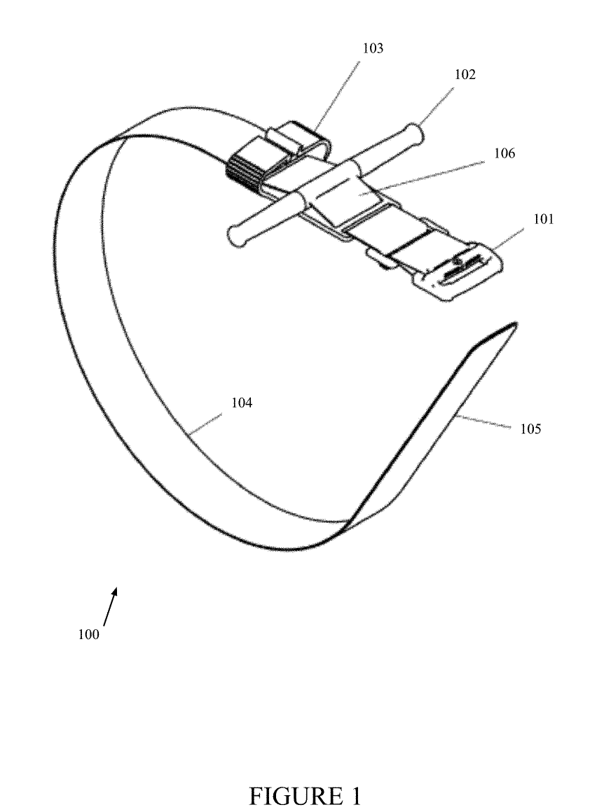

FIGS. 1 through 5, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged tourniquet assembly.

FIG. 1 illustrates windlass tourniquet 100 in accordance with an embodiment of the disclosure. Tourniquet 100 comprises buckle 101, tightening rod 102, C-shaped clip 103, main strap 104, and tensioning web 106. Main strap 104 comprises distal end 105, which is designed to be inserted through buckle 101 and to fold back onto main strap 104. Main strap 104, including distal end 105, may include hook-and-mesh closure strips (e.g., Velcro strips) on the surface so that distal end 105 attaches to main strap 104 when folded back onto main strap 104. As will be explained below in greater detail, rod 102 includes a writing tool (not shown) that is embedded in rod 102. The writing tool may be a pen, a pencil, a marker, or the like.

Tensioning web 106 is inserted through a slot in rod 102. Tensioning web 106 may be configured to be inserted internally in a slot or sleeve throughout the entire length of main strap 104 so that when rod 102 is twisted, tensioning web 106 is tightened and the tightening force is applied along the length of main strap 104, thereby constricting the tourniquet strap. Once tightened, rod 102 is inserted in the opening of clip 103 to prevent rod 102 from unwinding and loosening tourniquet 100.

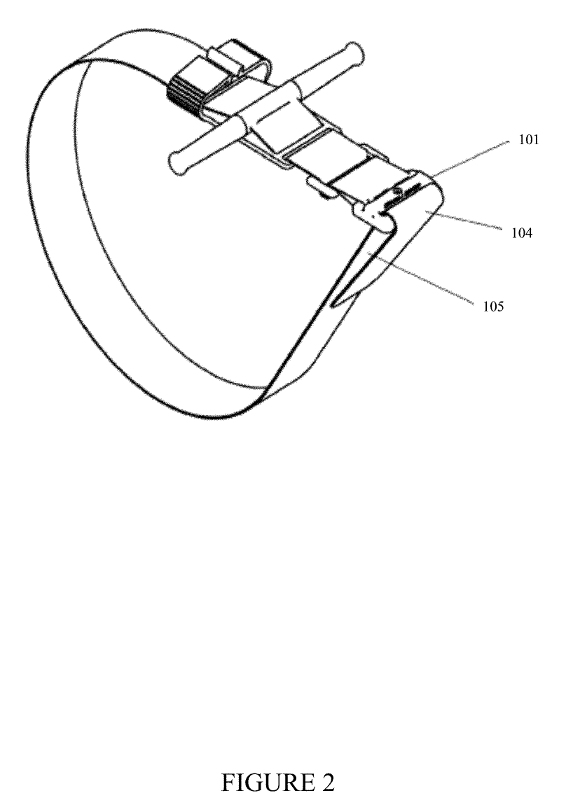

FIG. 2 illustrates tourniquet 100 in FIG. 1 in further detail. In FIG. 2, distal end 5 of main strap 104 is inserted through buckle 101, folded back onto main strap 104, and held in place by hook-and-mesh closure strips (e.g., Velcro strips) on the surfaces of distal end 105 and main strap 104. Tightening rod 102 is not yet twisted or rotated in FIG. 2 and tensioning web 106 is still slack. Thus, rod 102 is not shown inserted in C-shaped clip 103. In FIG. 2, tourniquet 100 forms a loop that encircles an injured limb.

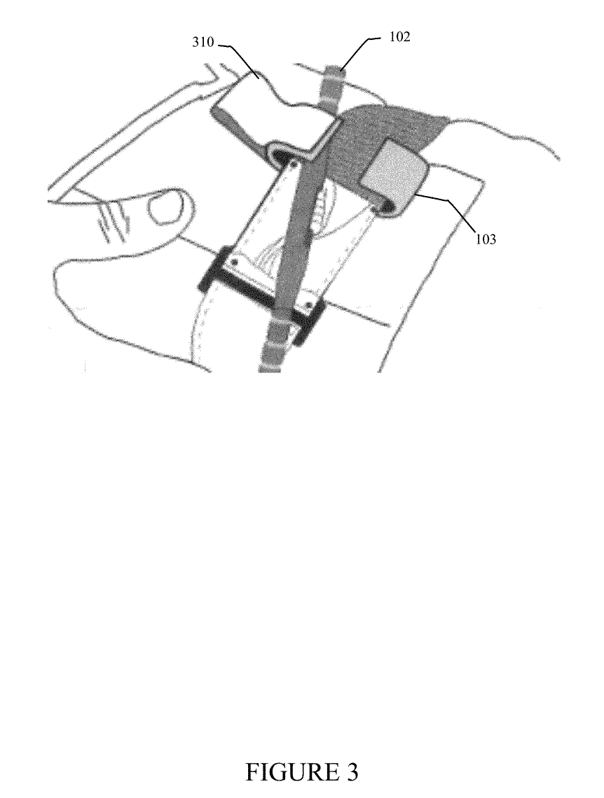

FIG. 3 illustrates tourniquet 100 applied to the limb of a patient. In FIG. 3, tightening rod 102 has been tightened and inserted into the opening of C-shaped clip 103 to prevent rod 102 from unwinding. In an advantageous embodiment, C-shaped clip 103 may include closure strip 310 that is closed over the opening of C-shaped clip 103 to prevent rod 102 from slipping out. Closure strip 310 may attach to C-shaped clip 103 by means of hook-and-mesh closure strips (e.g., Velcro strips) on the surfaces of closure strip 310 and C-shaped clip 103.

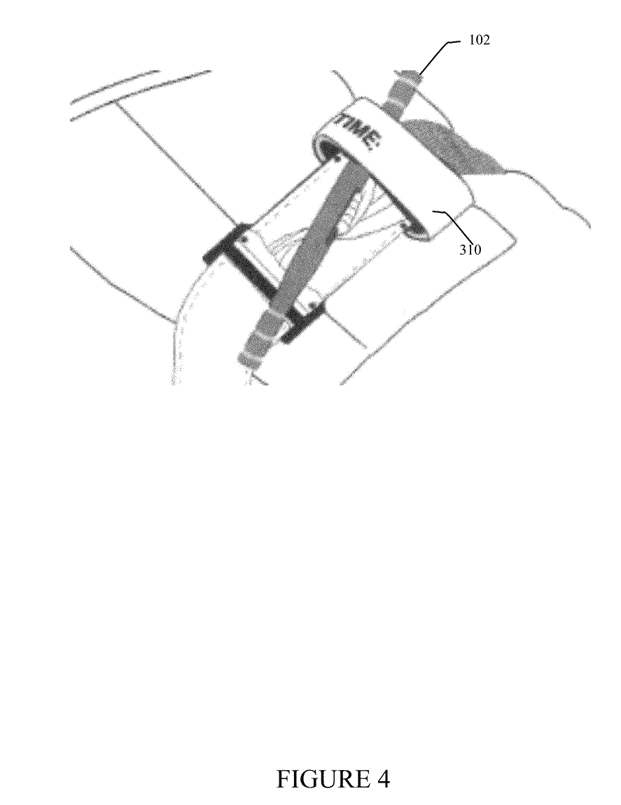

FIG. 4 illustrates tourniquet 100 in FIG. 3 in further detail. In FIG. 4, closure strip 310 has been closed across the opening of C-shaped clip 103, to thereby hold tightening rod 102 in place. Advantageously, the outer facing surface of closure strip 310 may be white or light in color and may include a label with the text “TIME:” printed thereon. Sufficient space is provided on the label to permit the person applying tourniquet 100 to write the time at which tourniquet 100 was applied to the injured limb.

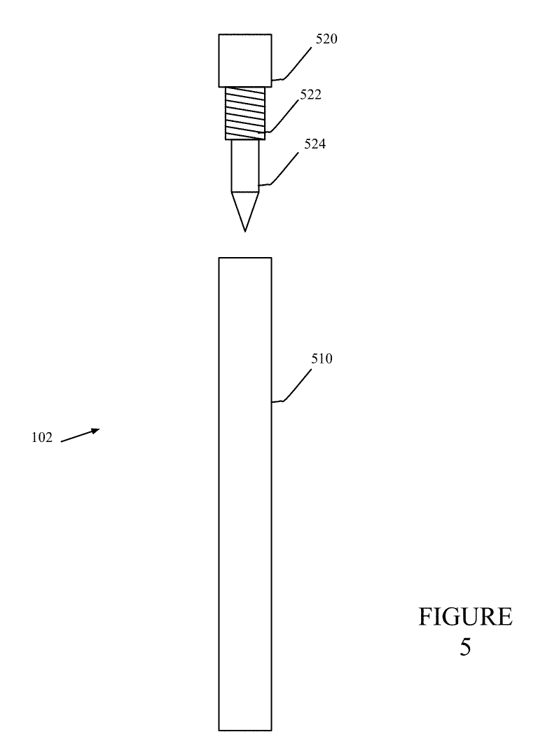

FIG. 5 illustrates in greater detail tightening rod 102 of exemplary tourniquet 100 in FIGS. 1-4 according to an embodiment of the disclosure. To ensure that the first responder or other person applying tourniquet 100 always has a writing tool available, tightening rod 102 comprises an internal writing tool that may be used to write the time on the label on closure strip 310. In exemplary embodiment, rod 102 is an elongated cylinder comprising two parts: main body 510 and first end portion 520. First end portion 520 includes threaded section 522 and writing tool 524.

Prior to use of tourniquet 100, first end portion 520 may be screwed into main body 510, which has internal threading (not shown) that match to threaded section 522. When tourniquet 100 is applied and tightened, the first responder or other person unscrews first end portion 520 from main body 510 and uses writing tool 524 to write the time at which tourniquet 100 was applied to the injured limb. First end portion 520 may then be re-inserted into main body 510 for later use. In an alternate embodiment, tightening rod 102 may include a second end portion (not shown) that includes a second writing tool (not shown) that may be inserted into main body 510 at the end of main body 510 opposite to first end portion 520. The inclusion of at least one writing tool, and perhaps two, in rod 102 ensures that a writing tool is always available.

In alternate embodiments, threaded section 522 may be omitted and replaced by another means of securing first end portion 510 within main body 510. For example, first end portion may snap into main body 510 by means of an annular indentation on the surface of first end portion 510 that engages a raised ridge or bump(s) within main body 510.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A tourniquet comprising:
a strap for encircling a limb; and
a tightening rod for tightening the strap around the limb, wherein the tightening rod includes a first writing tool.

2. The tourniquet as set forth in claim 1, wherein the first writing tool is disposed internally to the tightening rod.

3. The tourniquet as set forth in claim 1, wherein the tightening rod is cylindrical in shape and the first writing tool is disposed internally to the tightening rod and held in place by means of screw threading.

4. The tourniquet as set forth in claim 1, wherein the tightening rod further includes a second writing tool.

5. The tourniquet as set forth in claim 4, wherein the first and second writing tools are disposed internally to the tightening rod.

6. The tourniquet as set forth in claim 4, wherein the tightening rod is cylindrical in shape and the first writing tool is inserted in a first end of the tightening rod and held in place by means of screw threading.

7. The tourniquet as set forth in claim 6, wherein the second writing tool is inserted in a second end of the tightening rod opposite the first end and held in place by means of screw threading.
